(19) United States
(12) Patent Application Publication
(10) Pub. No.: US 2006/0153395 A1
(43) Pub. Date: Jul. 13, 2006

(54) Clinical assistant for cochlear implant care

(76) Inventors: Koen van Den Heuvel, Hove (BE); Jan Janssen, New South Wales (BE); Jochen Nicolai, New South Wales (BE); Geert Smits, New South Wales (BE)

Correspondence Address: JAGTIANI + GUTTAG, 10363-A DEMOCRACY LANE, FAIRFAX, VA, 22030, US

(21) Appl. No.: 10/537,027

(22) PCT Filed: Nov. 7, 2003
(86) PCT No.: PCT/AU03/01475

§ 371 Date: Jan. 5, 2006

(30) Foreign Application Priority Data
Nov. 7, 2002 (AU) 2002952675

(51) Int. Cl. H04R 29/00; A61B 5/00; A61B 5/12

(52) U.S. Cl. 381/60; 600/559; 73/585

(57) ABSTRACT

A system for performing one or more tests on a hearing prosthesis is provided. The system is usable by the recipient of the prosthesis, to reduce a testing load on a clinician. The system has a computer that processes software instructions and outputs signals in response to said instructions; a prosthesis interface that provides transfer of signals from the computer to the prosthesis and/or from the prosthesis to the computer; and an interface that allows the recipient of the prosthesis to at least partially control at least some aspect of the tests performed on the prosthesis that is interfaced with the computer.

### TECHNICAL FIELD

The present invention relates to a system for providing and delivering more efficient hearing implant care.

### BACKGROUND OF THE INVENTION

Over recent times, hearing prostheses, and in particular, hearing implants have become more widespread in their use as the benefits become more widely realised by the hearing impaired.

Hearing loss can be due to many different causes. One type of hearing loss is conductive hearing loss which occurs when the normal mechanical pathways for sound to reach the hair cells in the cochlea are impeded, for example, by damage to the ossicles. Conductive hearing loss may often be helped by use of conventional hearing aids, which amplify sound so that acoustic information does reach the cochlea and the hair cells.

In many people who are profoundly deaf, however, the reason for deafness is sensorineural hearing loss. This type of hearing loss is due to the absence of, or destruction of, the hair cells in the cochlea which transduce acoustic signals into nerve impulses. These people are thus unable to derive suitable benefit from conventional hearing aid systems, because there is damage to or absence of the mechanism for nerve impulses to be generated from sound in the normal manner.

Cochlear™ implant systems have been developed for persons with sensorineural hearing loss which bypass the hair cells in the cochlea and directly deliver electrical stimulation to the auditory nerve fibres, thereby allowing the brain to perceive a hearing sensation resembling the natural hearing sensation normally delivered to the auditory nerve. U.S. Pat. No. 4,532,930, the contents of which are incorporated herein by reference, provides a description of one type of traditional hearing implant system.

Typically, hearing implant systems consist of a microphone for detecting acoustic signals and a sound processor for transforming the acoustic signals, particularly speech, into patterns of electrical stimulation. The sound processor is typically worn externally, either behind the ear of the recipient or in a body worn pouch, and is programmed to meet the particular requirements of the recipient. The sound processor can include several different schemes for processing the acoustic signal and transforming the signal into electrical stimuli, with these schemes well known in the art. The electrical stimuli are then transferred, together with a power signal, to an implanted receiver/stimulator unit positioned within the head of the user. Traditionally, this transcutaneous transmission occurs via the external transmitter coil which is positioned to communicate with an implanted receiver coil provided with the receiver/stimulator unit. This communication serves two essential purposes, firstly to transcutaneously transmit the coded sound signal and secondly to provide power to the implanted receiver/stimulator unit. Conventionally, this link has been in the form of a radio frequency (RF) link, but other such links have been proposed and implemented with varying degrees of success. The implanted receiver/stimulator unit traditionally includes a receiver coil that receives the coded signal and power from the external processor component, and a stimulator that processes the coded signal and outputs a stimulation signal to an intracochlea electrode assembly which applies the electrical stimulation directly to the auditory nerve producing a hearing sensation corresponding to the original detected sound.

Due to the complex biophysical phenomena associated with the electrical excitation of neurons and the psychophysical phenomena regarding the interpretation of neural activity by the auditory nervous system, not all recipients have been found to benefit from the same speech processing strategy. In this regard, it has been found that the quality and intelligibility of speech percepts evoked by a hearing prosthesis may be improved in a given recipient by more specific manipulations of the electrical stimulus waveforms tailored to that recipient.

In this regard, it is important that when a recipient is first implanted with a hearing implant, in order for them to obtain the most benefit from the device, it needs to be adjusted/fitted to suit their specific needs. As the useful dynamic range for electrical stimulation is relatively narrow and varies across recipients and electrodes, there is a need to individually tailor the characteristics of electrical stimulation for each recipient. Simple psychophysical measurements establish the useful range for each electrode, and such parameters can be stored within the recipient's sound processor for continual use. This procedure is often referred to as “mapping” and is the term given to the process of measuring and controlling the amount of electrical current delivered to the cochlea. It is this process that ensures stimulation from the implant provides a recipient with comfortable and useful auditory percepts, and is essential in ensuring that the recipient receives maximum benefit from the hearing implant.

As the implant system is designed to present acoustic information, in particular speech, to a recipient in a usable form, the initial aim of the mapping process is to optimise the information provided for a particular recipient. The “mapping” process is a key part of the post-operative management of all hearing implant recipients and occupies a significant proportion of the post-operative clinical time.

As well as ensuring that the implant is fitted to the recipient immediately following implantation, it is also important to provide continual, ongoing management of the device. Typically, the initial fitting session is aimed at providing the recipient with the ability to experience sounds produced by the implant, so that they can become accustomed to the type of sounds experienced. After this stage, it is important that the sound information is optimised to ensure that the benefit obtained from the hearing implant is maximised. This is particularly important during the first three months following initial fitting as well as over the lifetime of the recipient at regular six month intervals or when the recipient requests an update or reports an issue.

Lifelong after-care for the recipient is essential in ensuring the continuing success of the implant and that the recipient obtains maximum benefit from the device. Such after-care includes; continual or regular monitoring of the operation of the device and comparison of current performance against historical records for that device; continual checking of the stability of the technical and physiological operation of the device; updating the device as new technology becomes available; ongoing technical service of the external parts of the system; distribution of spare parts and replacement batteries; as well as ongoing recipient counselling. Such follow-up sessions typically require standard checks of the recipient's threshold and comfort levels and the integrity of the overall system including speech processor and microphone.

During after-care, it is important that any problems are quickly and efficiently detected and remedied. If there are areas where the recipient is experiencing difficulty, or where tests suggest a problem, such as speech perception, it is important that this is identified and investigated as the growth and development of the recipient's communication skills may become affected. This is particularly important in relation to infants implanted with hearing implant systems to ensure that they can develop speech and communication skills in their early development phase.

Studies have shown that in some clinics that provide after-care to hearing implant recipients, this function alone can take up to 30% of the clinic's total hearing implant program workload. As the after-care function is provided mostly by audiologists, this maximisation of the audiologist's time directly competes with the time available for the audiologist to identify and work with potential new recipients who may benefit from a hearing implant.

Typically, much of the after-care requires dealing with relatively minor issues, Such as hardware defects and/or use of accessories or replacements. Such after-care does not necessarily require the attention of an audiologist as their time is best spent on adjusting a recipient's map or speech processing settings, in response to a change in hearing status.

Further, many recipients live reasonable distances from support clinics, which are mainly located in larger cities, and as such they may be dissuaded from visiting a clinic as often as a recipient living relatively close to a clinic. Such recipients may contact the clinic only when they have a specific problem which greatly affects the performance of their implant, rather than routinely contacting the clinic. This may result in such recipients not obtaining maximum benefit from their implant.

Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed before the priority date of each claim of this application.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

### SUMMARY OF THE INVENTION

According to a first aspect, the present invention provides a system for performing one or more tests on a hearing prosthesis, the system being usable at least in part by the recipient of the prosthesis, the system comprising:

a computer that processes software instructions and outputs signals in response to said instructions;

a prosthesis interface means that provides transfer of signals from said computer to the prosthesis and/or from the prosthesis to the computer; and

an interface that allows the recipient of the prosthesis to at least partially control at least some aspect of the tests performed on the prosthesis that is interfaced with the computer.

In preferred embodiments of the first aspect of the invention, the computer is a stand alone computer adapted to perform one or more tests on the hearing prosthesis.

In some embodiments of the first aspect of the present invention, the computer may be located in a clinic for use by the recipient when visiting the clinic. In such embodiments, the computer is preferably operable to provide results of the tests for immediate assessment by a clinician. The computer may additionally or alternatively be operable to store results of the tests for later assessment.

In alternate embodiments of the invention, the computer may be at a location remote from a clinician of the recipient, such as the recipient's home. In such embodiments, the computer is preferably operable to store results of the tests on a storage means for forwarding to the clinician of the recipient. The storage means may be in the prosthesis. Alternatively, the storage means may be a portable storage means such as a magnetic disc, to be physically forwarded to the clinician of the recipient.

In some embodiments of the first aspect of the invention, the computer may be operable to obtain software instructions from the hearing prosthesis.

In preferred embodiments of the first aspect of the invention, the computer is operable to deliver results of the test electronically to a computer of a clinician of the recipient, for example via the Internet.

According to a second aspect, the present invention provides a system for performing one or more tests on a hearing prosthesis, the system comprising:

a first computer that processes a set of software instructions and outputs signals in response to said instructions;

a prosthesis interface means that provides a transfer of signals between said first computer and the hearing prosthesis; and

a second computer that provides said set of software instructions to said first computer to control at least some aspects of the tests performed on the hearing prosthesis that is interfaced with said first computer.

In preferred embodiments of the second aspect of the invention, the first computer is located remotely from a clinician of the recipient, such as in a home of the recipient. In such embodiments, the first computer is preferably operable to deliver results of the test electronically to the second computer, such as via the Internet. In such embodiments the second computer is preferably operable to store electronically delivered results of the tests.

In preferred embodiments of the second aspect of the invention, the second computer is a computer of a clinician of the recipient.

In preferred embodiments of the second aspect of the invention, the second computer is operable to provide a set of instructions to the first computer instructing the first computer to perform one or a series of tests on the hearing prosthesis. In such embodiments, the second computer is preferably operable to generate the set of instructions to the first computer based on test results received from the hearing prosthesis. Additionally or alternatively, the second computer may be operable to modify the set of instructions to the first computer based on test results received from the hearing prosthesis.

In some embodiments of the second aspect of the present invention, the first computer may operate as a dumb terminal under the control of the second computer. Alternatively, the first computer may store test software, and may be operable to operate the test software upon receipt of a trigger signal from the second computer.

Embodiments of the second aspect of the present invention may further comprise a recipient interface interoperable with the first computer, enabling recipient control of some characteristics of tests carried out on the recipient's prosthesis. The recipient interface may comprise a graphical user interface displayed on a monitor screen of the first computer. The recipient interface may present instructions as to required steps to be performed by the recipient in carrying out a test. Such instructions may comprise instructions as to how the recipient may start or stop a test.

In preferred embodiments of the second aspect of the present invention, the second computer comprises an application enabling the recipient's clinician to configure an appropriate set of test instructions for transmission to the first computer. In such embodiments, the application may present a graphical user interface on a screen of the second computer to facilitate the recipient's clinician's configuration of the set of test instructions.

In embodiments of the second aspect of the present invention, the prosthesis interface means may comprise an external port of the first computer capable of being connected to the hearing prosthesis.

Further embodiments of the second aspect of the present invention may further comprise an input device to receive recipient control instructions.

According to a third aspect, the present invention provides a method of testing a hearing prosthesis, the method comprising:

providing a first computer that processes software instructions and outputs signals in response to said instructions;

transferring the signals from the first computer to the prosthesis; and

providing an interface that allows the recipient of the prosthesis to at least partially control at least some aspect of the tests performed on the prosthesis that is interfaced with the first computer.

In embodiments of the third aspect of the present invention, the first computer may be a stand alone computer. The first computer may be provided in a clinic for use by the recipient when visiting the clinic.

Preferred embodiments of the third aspect of the present invention further comprise the step of providing results of the tests for immediate assessment by a clinician. Additionally or alternatively, embodiments of the third aspect of the invention may further comprise the step of storing results of the tests for later assessment.

In preferred embodiments of the third aspect of the present invention, the first computer is provided at a location remote from a clinician of the recipient, such as at a home of the recipient. In such embodiments, the method of the third aspect of the present invention preferably further comprises storing results of the tests on a storage means and forwarding the storage means to the clinician of the recipient.

Embodiments of the third aspect of the invention may further comprise obtaining the software instructions from the hearing prosthesis.

Preferred embodiments of the third aspect of the present invention further comprise electronically transmitting results of the tests to a computer of a clinician of the recipient.

According to a fourth aspect, the present invention provides a method of performing one or more tests on a hearing prosthesis, the method comprising:

providing a first computer that processes a set of software instructions and outputs signals in response to said instructions;

transferring signals between said first computer and the hearing prosthesis; and providing a second computer that provides said set of software instructions to the first computer to control at least some aspect of the tests performed on the hearing prosthesis by the first computer.

In preferred embodiments of the fourth aspect of the present invention, the first computer is provided at a location remote from a clinician of the recipient, such as at a home of the recipient.

Preferred embodiments of the fourth aspect of the present invention further comprise the first computer electronically delivering results of the tests to the second computer. The first computer may electronically store results of the tests.

Preferred embodiments of the fourth aspect of the present invention further comprise the second computer providing a set of instructions to the first computer instructing the first computer to perform one or a series of tests on the hearing prosthesis. Such embodiments may further comprise the second computer generating said set of instructions to the first computer based on test results received from the hearing prosthesis. Additionally or alternatively, such embodiments may further comprise the second computer modifying the set of instructions to the first computer based on test results received from the hearing prosthesis.

In some embodiments of the fourth aspect of the invention, the first computer may operate as a dumb terminal under the control of the second computer. Alternatively, the first computer may store test software, and may operate said test software upon receipt of a trigger signal from the second computer.

Preferred embodiments of the fourth aspect of the invention further comprise providing a recipient interface interoperable with the first computer, enabling recipient control of some characteristics of tests carried out on the recipient's prosthesis. In such embodiments, the recipient interface may comprise a graphical user interface displayed on a screen of the first computer. Further, the recipient interface may present instructions as to required steps to be performed by the recipient in carrying out a test. The instructions may comprise instructions as to how the recipient may start or stop a test.

In preferred embodiments of the fourth aspect of the invention, the method further comprises a clinician configuring a set of test instructions by use of an application of the second computer, for transmission to the first computer. Such embodiments preferably further comprise the application of the second computer presenting a graphical user interface on a screen of the second computer to facilitate the clinician's configuration of the set of test instructions.

In preferred embodiments of the fourth aspect of the present invention, the method further comprises providing an input device to receive recipient control instructions.

In accordance with the present invention, the computer can in one embodiment be adapted to stand alone and perform said one or more tests on the hearing prosthesis. In such an embodiment, the computer may be housed in a clinic of the clinician and used by the recipient when visiting their clinician. The results of the tests can be assessed immediately by the clinician or stored for assessment at some later time that is perhaps more convenient for the clinician.

In another embodiment, the computer can be at a location remote from the recipient's clinician. In a preferred such embodiment, the computer is housed in the recipient's home or at a location that is relatively more convenient for the recipient to access than the clinic. In such embodiments, the results of the tests can be stored in a storage means in the hearing prosthesis or can be stored in a suitable storage means used in conjunction with the computer. For example, the results can be saved on a portable storage means, such as a disc, and then provided to a clinician responsible for managing the after-care of the recipient of the prosthesis. The stored results may be retained by the recipient and only provided to the clinician on the recipient's next visit to the clinician or can be posted to the clinician using normal mail services.

In a further embodiment, the clinician can set up the configuration for one or more tests in the hearing prosthesis. When the recipient decides or is instructed to test their prosthesis, the prosthesis can be interfaced with the computer, with the computer being adapted to read the test configuration from the hearing prosthesis.

In another embodiment, the computer is adapted to deliver the results electronically to a computer used by the clinician. Such electronic transmission is preferably provided by the Internet but other suitable modes of transmission can be envisaged. In this embodiment, the clinician's computer is preferably remote from the first computer that is interfaced to the hearing prosthesis. The clinician's computer can preferably store the results delivered from the first computer for review and assessment by the clinician at a convenient time.

The clinician's computer can further be adapted to interact with the first computer. In this embodiment, the clinician's computer can provide a set of instructions to the first computer instructing the first computer to perform one or more tests on the hearing prosthesis. The instructions of the clinician's computer can be based on or modified depending on the results received from the first computer. In one embodiment, the first computer can be essentially a dumb terminal and act mainly as a means of delivering the instructions from the clinician's computer to the prosthesis. In a more preferred embodiment, the first computer simply requires a trigger signal from the clinician's computer which then activates appropriate software already loaded on the first computer which results in the first computer performing one or more desired tests on the prosthesis.

In preferred embodiments of the present invention, the first computer can be housed at a location remote from the clinician. In a preferred embodiment, the first computer is housed in the recipient's home or at a location that is relatively more convenient for the recipient to access than the clinic. The first computer is preferably adapted to deliver the results electronically to the second computer that is preferably used by the clinician. Such electronic transmission is preferably provided by the Internet but other suitable modes of transmission can be envisaged. The second computer can preferably store the results delivered from the first computer for review and assessment by the clinician at a convenient time.

The second computer can further be adapted to interact with the first computer. In this embodiment, the second computer can provide a set of instructions to the first computer instructing the first computer to perform one or a series of tests on the hearing prosthesis. The instructions of the second computer can be generated based on the results received from the first computer or modified depending on the results received from the first computer. In one embodiment, the first computer can be essentially a dumb terminal and act mainly as a means of delivering the instructions received from the second computer. In a more preferred embodiment, the first computer simply requires a trigger signal from the second computer, the trigger signal activating appropriate software already loaded and stored on the first computer which results in the first computer performing one or more desired tests on the prosthesis.

The system further preferably comprises a recipient-to-first computer interface that allows the recipient of the prosthesis to at least partially control at least some aspect of the tests performed on a prosthesis that is interfaced with the computer.

The recipient interface device preferably comprises a graphical user interface displayed on a screen, a keyboard, a keypad, and/or a pointing device, such as a mouse or stylus. For example, the graphical user interface preferably displays a number of messages to the recipient. These messages can include instructions as to required steps to be performed by the recipient in carrying out the test. The graphical user interface also preferably provides messages advising the recipient how to commence and/or stop a test. A test can typically be started or stopped by pressing any key of the computer's keyboard or by clicking on an icon displayed on the graphical user interface using the pointing device.

The hearing prosthesis is preferably a cochlear™ implant. For the purposes of the description herein, the systems will be described more fully in relation to their preferred use in testing cochlear™ implants. It will, however, be appreciated that the systems could be used to perform tests on hearing aids or other hearing implants.

The second computer is preferably loaded with appropriate software that enables the clinician operating this computer to configure an appropriate set of instructions that can be sent to the recipient's computer over the appropriate network connection. To allow this, the second computer is also preferably provided with a graphical user interface that enables the clinician to configure a series of tests to measure desired aspects of the recipient's cochlear™ implant system. The interface of the second computer again preferably comprises a display provided on a screen that can be manipulated or controlled by the clinician using a keypad, keyboard and/or a pointing device, such as a mouse.

The prosthesis interface means can comprise an external port of the first computer capable of being connected via a cable to the external speech processor of the hearing implant of the recipient. The cable may be a universal serial bus (USB) cable, a cable in accordance with standard IEEE1394 (eg FireWire), or the like. It is envisaged that the speech processor would during most tests be mounted so as to be in signal communication with the implanted stimulator unit of the implant, thereby providing complete access of the system to the hearing implant.

In a further embodiment, the first computer can also be provided with a custom input device for use by the recipient. Such a device may include a series of buttons and/or lights that require manipulation by the recipient during testing of the hearing implant.

Upon delivery of the set of instructions to the first computer, the recipient can preferably at least partially control the instructions by reading the messages displayed on the screen and responding appropriately. The screen, for example, may display a message asking the recipient to perform a series of tasks. The computer then collects relevant information resulting from the completion of such tasks.

Once the one or more tests have been completed, a set of results collected during the execution of the tests is then preferably sent back to the clinician's computer for evaluation by the clinician. In this embodiment, the clinician's computer can perform an appropriate evaluation of the data received and assess it against predefined criteria set by the clinician. The results of the assessment can then be conveyed to the clinician who is then in a position to make an informed decision on what, if any, subsequent action can be made.

The step of evaluating the received results can have a number of outcomes, depending upon the results of the evaluation. Should the evaluation show that the performed tests were passed, no further action will be required by the recipient and the recipient would normally be informed of this. However, should the results of the tests indicate a problem with the hearing implant that requires corrective action, the recipient would be informed of the need to visit the clinic/hospital and an appointment could be made and confirmed by the recipient for such a session. With the present invention, it is also possible that should the test results be inconclusive, the clinician could configure further tests to ascertain the function of the hearing implant device.

The screen of the clinician's computer can output a first display that assists the clinician in configuring the appropriate set of instructions for delivery to the recipient's computer. This first display allows the clinician to enter the details of a recipient or select a recipient from a database of recipients and also to view the recipient's detailed data and history of past tests performed, to establish whether there are any specific issues that require testing.

Once the clinician has selected the recipient and considered their history, they must then configure appropriate tests to be undertaken to assess the performance and the operation of the hearing implant. In a further embodiment, the displays provided on the clinician's computer and/or the recipient's computer can display messages in the preferred language suitable for the specific recipient. The software preferably allows ready changing of the language of the messages displayed by one or both computers.

In a preferred embodiment of the invention, once the clinician has accessed the first display, the clinician has three options. They can create a new test configuration, open a template test configuration, or open a recipient test configuration. This decision will be largely dependant upon the knowledge the clinician possesses of the recipient's hearing implant system.

It is considered that in most instances, the clinician will open a template test configuration which preferably provides a standard template for configuring the tests to suit the chosen recipient.

Should the clinician have a previous test template stored that has been configured for the particular recipient, the recipient test configuration would normally be chosen. Alternatively, should the clinician wish to construct a new test configuration different to that of the template, the option to create a new test configuration will be selected.

Following this step, the tests need to be configured and this can be done by manipulating the template displayed on the clinician's computer. By doing this, the clinician is able to run one or more standard tests checking a number of features of the hearing implant system. These could be:

(i) a standard system integrity test to ensure that all the components of the system are operating correctly;

(ii) a neural response threshold test to compare current measured neural response thresholds with previously measured thresholds;

(iii) threshold (T) stimulation level and comfortable (C) stimulation level test to determine whether the correct dynamic range of stimulation is set for each electrode; and

(iv) tests to check loudness balancing, pitch ranking and phoneme discrimination to give the clinician an indication of the effectiveness of the implant.

The clinician can also select which stored maps are to be used in the testing.

Alternatively, in this step the clinician is able to select an advanced configuration to customise the standard tests to suit the specific needs of the recipient. In this regard, the clinician could modify the input device used, the specific electrodes used in the threshold level tests, the acceptable difference allowable between measured results and stored data, the order of tests to be performed, and the samples taken for the speech tests. Once the clinician has selected such a configuration, the clinician can then alter specific aspects of the tests to suit the needs of the recipient.

Once all the tests have been configured and set for the specific recipient, the clinician can save the configuration for remote execution, to be sent to the recipient's computer.

Should the recipient be present with the clinician at the time of configuration, the clinician can also start testing the recipient by starting the recipient's interface upon completion of the configuration session. In this instance, the clinician can check whether the speech processor (SP) is connected to the computer by running a speech processor connection test, to ensure that the test collects desired results.

Following delivery of the configured tests at the recipient's computer, the recipient runs the test via an appropriate interface, such as has been defined herein.

The recipient's computer takes the recipient through a series of tests in sequence, as determined by the clinician, collecting the data and results of each test. After each test is performed, the measured results are merged into a results file where this file is then used by the clinician's computer following the completion of the recipient session. This file can be exported to the clinician in a number of ways, for example the file can be sent via the Internet or LAN connection to the recipient where it is received by the clinician application and acted upon. Or it can be stored in the recipient's speech processor and accessed from the speech processor at the recipient's next visit to the clinician.

An example configuration file is shown below:

```
<?xml version=“1.0” encoding=“utf-8” ?>
<Configuration>
  <PatientID>AEB182FC-F435-4f7e-85AE-D0D262464D86</PatientID>
  <TestID>B7316444-052D-464c-BC68-DC6474B822AA</TestID>
  <TestItems>
```

<Test1>

<Name>Impedance Test</Name>
<Channels>

<Channel>1</Channel>
<Channel>5</Channel>

</Channels>
<Modes>

<Mode>CG</Mode>
<Mode>MP1</Mode>

</Modes>

</Test1>
<Test2>

<Name>Threshold Level Measurement</Name>
<Map>1</Map>
<Channels>

<Channel>1</Channel>
<Channel>5</Channel>
<Channel>10</Channel>

</Channels>

</Test2>

</TestItems>
</Configuration>

This example configures an impedance test (test 1) and a threshold level measurement test (test 2) on the recipient application. As can be seen, the impedance test is carried out on channels 1 and 5 of the recipient's hearing implant in both common ground (CG) and monopolar 1 (MP1) mode. The threshold level measurement test is carried out on channels 1, 5 and 10. Each of these parameters are set by the clinician when configuring the test.

Based upon this configuration file shown above, an example results file generated is also shown below.

```
<?xml version=“1.0” encoding=“utf-8” ?>
<Result>
  <PatientID>AEB182FC-F435-4f7e-85AE-D0D262464D86</PatientID>
  <TestID>B7316444-052D-464c-BC68-DC6474B822AA</TestID>
  <TestItems>
```

<Test1>

<Name>Impedance Test</Name>
<Impedance>

<Mode>CG</Mode>
<Channel>1</Channel>
<Value>10500</Value>

</Impedance>
<Impedance>

<Mode>CG</Mode>
<Channel>5</Channel>
<Value>10560</Value>

</Impedance>
<Impedance>

<Mode>MP1</Mode>
<Channel>1</Channel>
<Value>10900</Value>

</Impedance>
<Impedance>

<Mode>MP1</Mode>
<Channel>5</Channel>
<Value>10800</Value>

</Impedance>

</Test1>
<Test2>

<Name>Threshold Level Measurement</Name>
<Level>

<Channel>1</Channel>
<Value>105</Value>

</Level>
<Level>

<Channel>5</Channel>
<Value>115</Value>

</Level>
<Level>

<Channel>10</Channel>
<Value>106</Value>

</Level>

</Test2>

</TestItems>
</Result>

As is shown above, the results of the configured tests, test 1 and test 2, are merged into this results file.

Following collection of the data for each test, the results data is preferably then sent back to the clinician for evaluation and decision on what course of action is required.

Once the recipient has received the configured test from the clinician, the recipient's computer preferably displays a personal welcome page to the recipient wherein the recipient can be satisfied that the test is specifically designed for their own needs. The recipient can control commencement of the test by performing an action requested by the computer, for example by clicking an icon having the word “Next” using a mouse pointer. On doing this, the recipient is preferably informed that the system will perform a system integrity check, to check that each aspect of the system is working correctly. The recipient is warned before any testing and in order for the test to be performed the recipient must elect to proceed with the test by performing an action requested by the computer, such as clicking on an icon having the word “Go” using a mouse pointer.

Should the recipient experience any difficulties or discomfort during testing, they have the ability to stop the testing at any time by pressing any button or “Stop”. This feature is similar to what would occur in a regular clinical visit, where the recipient could indicate to the clinician any issues and the clinician would then stop the testing. However the present invention provides for such functionality without requiring the presence of the clinician.

Once the recipient has started the system integrity test, the recipient is informed by a message on the screen that the test is being performed, and the recipient is provided with an indication of the progress of the test. Upon completion of the test, the recipient is informed by a message on the screen that the test has been completed and is asked to proceed to the next stage in the process. Again, the recipient preferably has control of commencement of the next stage of the test.

At the completion of the system integrity test, the recipient application preferably undergoes a basic test that checks whether the system integrity test has found any severe system integrity problems. Should such problems be found, the application would inform the recipient of this and suggest that the recipient contact the clinician for further attention. This would then normally bring a halt to the testing process, as the presence of system integrity problems would typically reduce the benefit of obtaining any further test results.

Following the successful completion of the system integrity test, the recipient is then preferably informed of the next test in the process, such as a psychophysics test. Upon commencement of this test, the recipient is given instructions on how the test will be conducted and direction as to how input is required from the recipient. Typically, the input will be in the form of using a mouse to activate the appropriate icon on the computer screen. For example, the recipient can be asked to press an icon having the word “YES” when they hear a tone and press an icon having the word “NO” when they do not hear anything. Once this test is complete, the recipient is preferably informed of this and asked to proceed to the next step in the process.

When the recipient has completed all tests configured for them, they are preferably informed of this and the application is closed. The recipient preferably clicks an icon having the word “End” to close the programme on their computer.

As discussed, upon completion of the configured tests, the results are preferably sent to the clinician for evaluation. This evaluation is performed by the clinician application which accesses the results file and tests the results in relation to previous or desired results.

Upon an evaluation of the results as discussed above for all the tests undertaken, the clinician is preferably provided with an interface on the clinician's computer providing an overview of the results. The clinician can then decide to view a detailed report of the test. Such a detailed report can show the measured values obtained in the test against the mapped values, or previously measured values in both a data form and a graphical form.

Based upon the results, the clinician can decide to proceed in a number of ways. Obviously, if the process indicated that all tests were passed, no specific action would be required and the clinician would inform the recipient of this and ask the recipient to repeat the test in, for example, 6 months time. Should there be some inconsistencies in the results, the clinician may wish to see the recipient personally or send the recipient further tests to undertake to determine whether the inconsistency is merely an error in recordings or is a concern that requires further monitoring.

As can be appreciated, the present invention provides a system which enables faster, more user friendly and less skill-intensive follow-up care and management of a hearing implant. This system allows for personal expert care from clinicians and audiologists without the need for onerous clinician visits, and allows the recipient to interact with the clinician from their own home or school.

The present invention preferably provides a system that allows for quicker and more efficient follow-up care of recipients of hearing implants that enables a full, standard and consistent service to be provided that increases the productivity of audiologists during clinical time; increases the quality of the follow-up service; standardises the results of follow-up tests; and reduces the number of erroneously diagnosed problems.

The present invention also preferably provides to the recipients of hearing implants increased quality of follow-up tests; a sense of control and participation in the monitoring of the performance of their device; and a system which allows for follow-up services to be provided in a friendlier environment, such as in the recipient's home or in a hearing centre.

It is considered that by providing an after-care system that is more efficient and user friendly as well as less audiologist-dependent, a more cost effective and attractive hearing implant management process is able to be implemented.

The present invention also preferably provides a system for use by recipients in follow-up visits that provides a relatively easy to use method of conducting Such tests.

### BRIEF DESCRIPTION OF THE DRAWINGS

By way of example only, preferred embodiments of the invention are now described with reference to the accompanying drawings, in which:

FIG. 1 is a view of one embodiment of the present system;

FIG. 1a illustrates one embodiment of a computer system for use in the present invention;

FIG. 2 is a flow diagram representing the overall process of an embodiment of the present invention;

FIG. 3 is a flow diagram representing the process associated with the clinical application of an embodiment of the present invention;

FIG. 4 is a representation of an embodiment of a clinical interface of the present invention;

FIG. 5 is an embodiment of an advanced clinical interface of the present invention;

FIG. 6 is a flow diagram of one embodiment of the recipient application of the present invention;

FIGS. 7-14 depict an example of one embodiment of a test as performed by the recipient;

FIG. 15 shows an embodiment of an evaluation of a test process according to the present invention; and

FIG. 16 shows an embodiment of an advanced evaluation report generated as a result of the present invention.

### BEST MODE FOR CARRYING OUT THE INVENTION

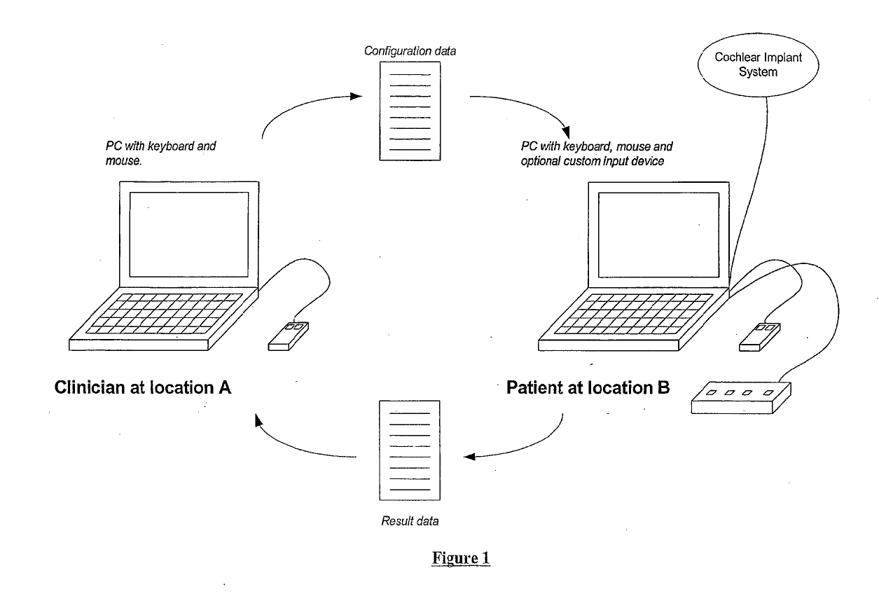

The invention is generally embodied on a computer system including one or more computers, such as the set-up as shown in FIG. 1. In the depicted embodiment, two stand alone personal computers are provided, with one computer 2 being for use by a clinician and the other computer 4 being for use by a recipient. These computers can be located in positions remote from each other and can be connected via an appropriate network connection, such as a LAN (Local Area Network) or via the Internet. In this regard, the computers can be located in the same room, such as a clinician's room; in the same building, such as in the same clinic or hospital; or in separate buildings or locations, with, for example, computer 2 being in a clinic and computer 4 being at the recipient's home, school or workplace.

While two computers are depicted, it will be appreciated that it is also possible for the present invention to be performed on one computer that is used by both the clinician and the recipient at different times. In another embodiment, the present invention could be performed using a single computer. In a further embodiment, the clinician can set up the configuration for one or more tests in the hearing prosthesis. When the recipient decides or is instructed to test their prosthesis, the prosthesis can be interfaced with a computer that is adapted to read the test configuration from the hearing prosthesis. This computer can be a stand-alone computer and can belong to the recipient, the clinician or a third party.

As is shown in FIG. 1, the clinician's computer 2 is a general stand-alone personal computer having a screen, keyboard and mouse. This computer is loaded with appropriate software according to the present invention to enable the clinician operating the computer to configure an appropriate recipient application 6 that can be sent to the recipient's computer 4 over the appropriate network connection. This is done by providing the clinician with an interface that enables the clinician to configure a series of tests to measure desired aspects of the recipient's hearing implant system. This will be described in more detail below. Once the clinician has completed configuring the desired tests the application 6 is then sent to the recipient's computer 4.

The recipient's computer 4, like the clinician's computer 2, is a general stand-alone personal computer having a screen, keyboard and mouse as well as connections to communicate with the recipient's hearing implant system. This could be in the form of a simple socket capable of being connected to the recipient's external speech processor, which is in turn connected to the implanted stimulator unit, thereby providing complete access of the system to the hearing implant.

As depicted, the computer 4 can also be provided with a custom input device 8 which may include a series of buttons and/or lights that require manipulation by the recipient during testing of the hearing implant.

Upon delivery of a recipient application 6, the recipient can then run the application on the computer 4 and follow the instructions of the application. In this regard, the computer 4 is provided with application software including a recipient interface that receives the recipient application 6 and performs the required tasks and communicates this to the recipient during execution. This interface may ask the recipient to perform a series of tasks and collects the relevant information resulting from the completion of such tasks.

Once the recipient application 6 has been completed, the result data 9 collected during the execution of the recipient application 6 is then sent back to the clinician's computer 2 for evaluation by the clinician. In this regard, the clinician's computer 2 performs an appropriate evaluation of the data received and assesses it against predefined criteria set by the clinician. The results of the assessment is then conveyed to the clinician who is then in a position to make an informed decision on what, if any, subsequent action can be made.

In more detail, the clinician's computer 2 and the recipient's computer 4 are preferably implemented by a conventional general-purpose computer 100, such as the one shown in FIG. 1a, wherein the processes described herein with reference to the Figures may be implemented as software executing on such a computer. In particular, the steps of a method in accordance with the present invention may be effected by instructions in the software that are carried out by the computer. The software may be divided into parts; for example comprising one part for generating instructions to the hearing prosthesis and receiving test results from the prosthesis, and another part to implement the user interface.

The software may be stored in a computer readable medium, including the storage devices described below, for example. In such embodiments, the software is loaded into the computer from the computer readable medium, and then executed by the computer. A computer readable medium having such software or computer program recorded on it is a computer program product. The use of the computer program product in the computer preferably effects an advantageous apparatus for performing one or more tests on a hearing prosthesis, the system being usable at least in part by the recipient of the hearing prosthesis, in accordance with embodiments of the invention.

Referring again to FIG. 1a, the computer system 100 comprises a computer 102, a video display 116, and input devices 118, 120. In addition, the computer system 100 can have any of a number of other output devices including line printers, laser printers, plotters, and other reproduction devices connected to the computer 102. The computer system 100 can be connected to one or more other computers via a communication interface 108c using an appropriate communication channel 130 such as a modem communications path, a computer network, or the like. The computer network may include a local area network (LAN), a wide area network (WAN), an Intranet, and/or the Internet

The computer 102 itself comprises a central processing unit(s) (simply referred to as a processor hereinafter) 104, a memory 106 which may include random access memory (RAM) and read-only memory (ROM), input/output (IO) interfaces 108a, 108b & 108c, a video interface 110, and one or more storage devices generally represented by a block 112 in FIG. 1a. The storage device(s) 112 can comprise one or more of the following: a floppy disc, a hard disc drive, a magneto-optical disc drive, CD-ROM, magnetic tape or any other of a number of non-volatile storage devices well known to those skilled in the art. Each of the components 104 to 112 is typically connected to one or more of the other devices via a bus 114 that in turn can consist of data, address, and control buses.

The video interface 110 is connected to the video display 16 and provides video signals from the computer 102 for display on the video display 116. User input to operate the computer 102 can be provided by one or more input devices 108b. For example, an operator can use the keyboard 118 and/or a pointing device such as the mouse 120 to provide input to the computer 102.

The system 100 is simply provided for illustrative purposes and other configurations can be employed without departing from the scope and spirit of the invention. Exemplary computers on which the embodiment can be practiced include IBM-PC/ATs or compatibles, one of the Macintosh (™) family of PCs, Sun Sparcstation (™), or the like. The foregoing are merely exemplary of the types of computers with which the embodiments of the invention may be practiced. Typically, the processes of the embodiments, described hereinafter, are resident as software or a program recorded on a hard disk drive (generally depicted as block 112 in FIG. 1a) as the computer readable medium, and read and controlled using the processor 104. Intermediate storage of the program and pixel data and any data fetched from the network may be accomplished using the semiconductor memory 106, possibly in concert with the hard disk drive 112.

In some instances, the program may be supplied to the user encoded on a CD-ROM or a floppy disk (both generally depicted by block 112), or alternatively could be read by the user from the network via a modem device connected to the computer, for example. Still further, the software can also be loaded into the computer system 100 from other computer readable medium including magnetic tape, a ROM or integrated circuit, a magneto-optical disk, a radio or infra-red transmission channel between the computer and another device, a computer readable card such as a PCMCIA card, and the Internet and Intranets including email transmissions and information recorded on websites and the like. The foregoing are merely exemplary of relevant computer readable mediums. Other computer readable mediums may be practiced without departing from the scope and spirit of the invention.

The method of testing the hearing prosthesis may alternatively be implemented in dedicated hardware such as one or more integrated circuits performing the functions or sub functions of the testing process. Such dedicated hardware may include graphic processors, digital signal processors, or one or more microprocessors and associated memories.

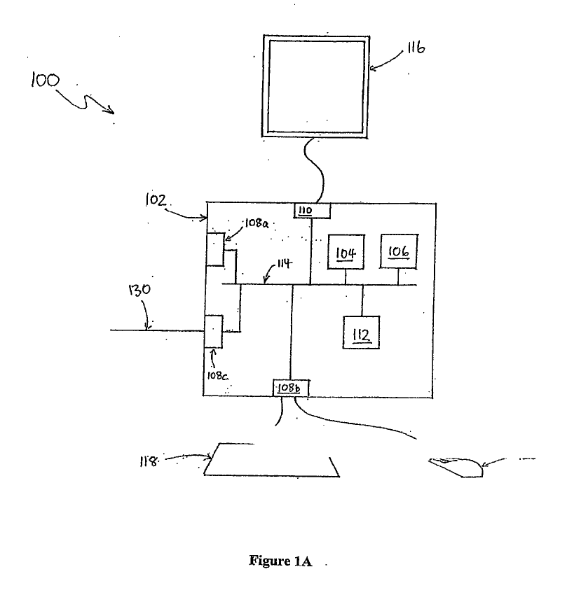

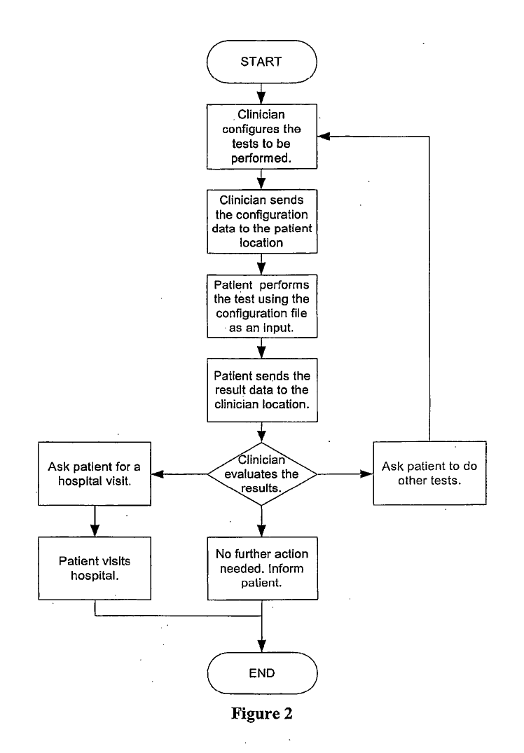

The testing process of an embodiment of the present invention is shown in FIG. 2. As is shown, the step of evaluating the received results can have a number of outcomes, depending upon the results of the evaluation. Should the evaluation show that the performed tests were passed, no further action will be required by the recipient and the recipient would be informed of this. However, should the results of the tests indicate a problem with the hearing implant that requires corrective action the recipient would be informed of the need to visit the clinic/hospital and an appointment could be made and confirmed by the recipient for such a session. With the present invention, it is also possible that should the test results be inconclusive, the clinician could configure further tests to ascertain the function of the hearing implant device.

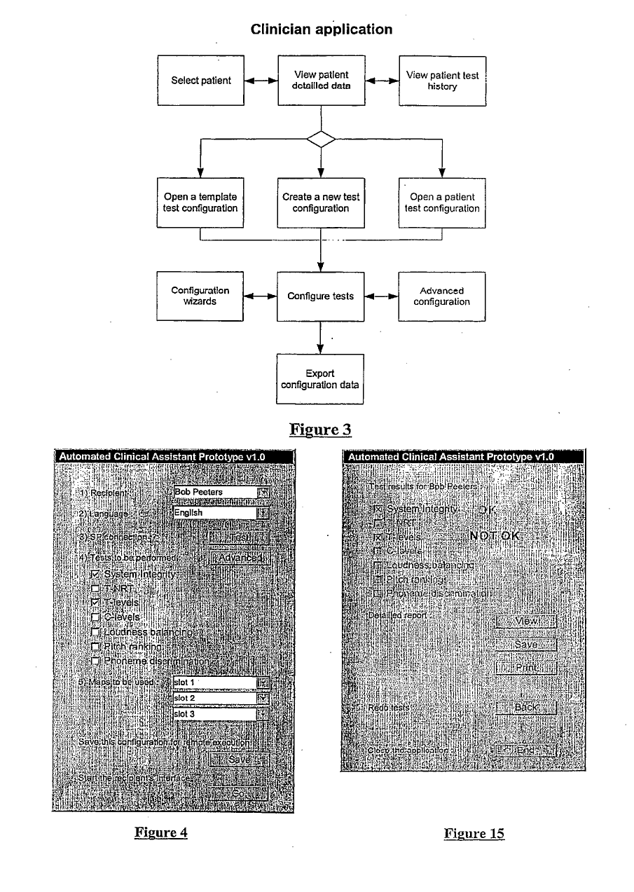

The step of configuring the recipient application is now described in more detail with reference to FIG. 3. In this regard, an interface such as that shown in FIG. 4 is displayed on the screen of the clinician's computer 2 to assist the clinician in configuring the appropriate application for the recipient's hearing implant. This interface allows the clinician to select a recipient from a database of recipients and also to view the recipient's detailed data and history of past tests performed, to establish whether there are any specific issues that require testing.

Once the clinician has selected the recipient and considered their history, they must then configure appropriate tests to be undertaken to assess the performance and the operation of the hearing implant. As shown in FIG. 4, the present invention can be used in countries with differing languages with the clinician being able to select the preferred language suitable for the specific recipient.

Once the clinician has accessed the display depicted in FIG. 4, the clinician has three options. They can create a new test configuration, open a template test configuration, or open a recipient test configuration. This decision is largely dependant upon the knowledge the clinician possesses of the recipient's hearing implant system.

It is considered that in most instances, the clinician will open a template test configuration such as that shown in FIG. 4, which provides a standard template for configuring the tests to suit the chosen recipient. Should the clinician have a previous test template stored that has been configured for the particular recipient, the recipient test configuration would normally be chosen. Alternatively, should the clinician wish to construct a new test configuration different to that of the template, the option to create a new test configuration will be selected.

Following this step, the tests need to be configured and this can be done by manipulating the template as shown in FIG. 4. By doing this, the clinician is able to run standard tests checking a number of features of the hearing implant system. These could be:

(i) a standard system integrity test to ensure that all the components of the system are operating correctly;

(ii) a neural response threshold test to compare current measured neural response thresholds with previously measured thresholds;

(iii) threshold stimulation level and comfortable stimulation level test to determine whether the correct dynamic range of stimulation is set for each electrode; and

(iv) tests to check loudness balancing, pitch ranking and phoneme discrimination to give the clinician an indication of the effectiveness of the implant.

As is shown, the clinician can also select which stored maps are to be used in the testing.

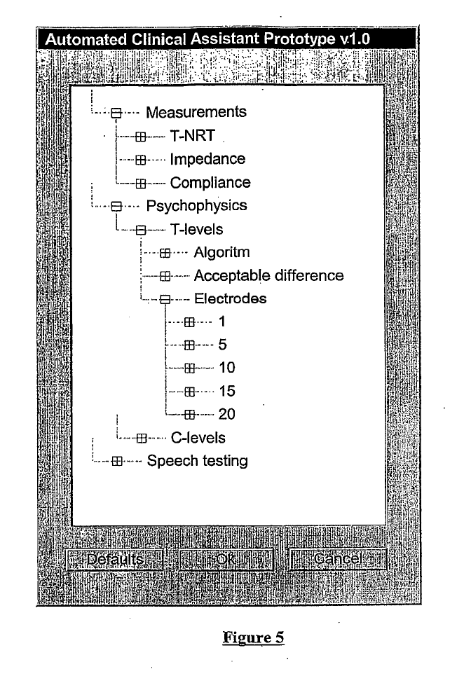

Alternatively, in this step the clinician is able to select an advanced configuration to customise the standard tests to suit the specific needs of the recipient. In this regard, the clinician could modify the input device used, the specific electrodes used in the threshold level tests, the acceptable difference allowable between measured results and stored data, the order of tests to be performed, and the samples taken for the speech tests. Once the clinician has selected the advanced configuration, as shown in FIG. 5, the clinician can then alter specific aspects of the tests to suit the needs of the recipient.

Once all the tests have been configured and set for the specific recipient, the clinician can save the configuration for remote execution, to be sent to the recipient's computer 4.

As is also shown in FIG. 4, should the recipient be present with the clinician at the time of configuration, the clinician can also start testing the recipient by starting the recipient's interface upon completion of the configuration session. In this instance, the clinician can check whether the speech processor (SP) is connected to the computer 4 by running the SP connection test, to ensure that the test collects desired results.

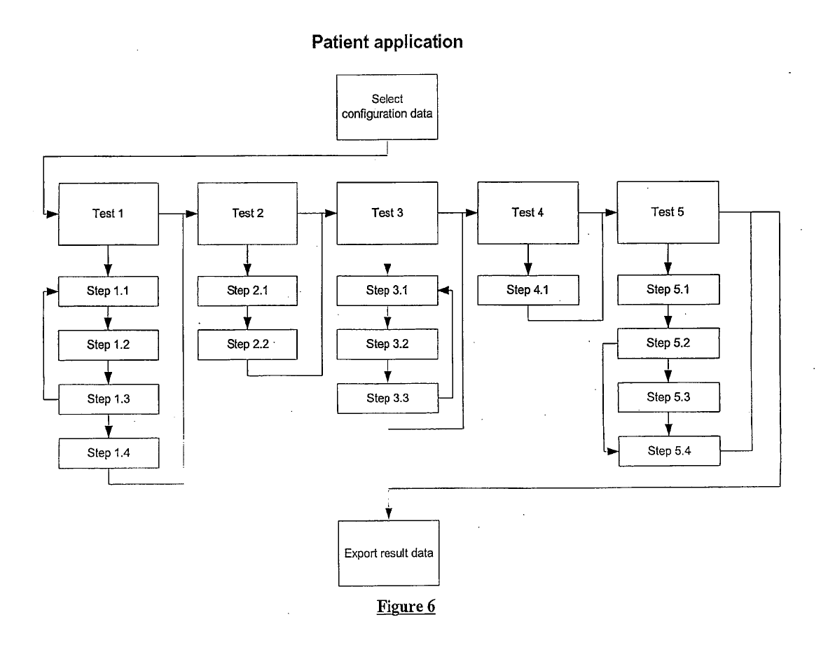

FIG. 6 depicts the process associated with the recipient receiving and performing the configured test discussed in FIGS. 4 and 5. As is shown, following delivery of the configured tests at the computer 4, the recipient runs the test via an appropriate interface.

As is shown, the interface then takes the user through a series of tests in sequence, as determined by the clinician, collecting the data and results of each test. Tests 1-5 as shown in FIG. 6 each configure a test module in the recipient application. After each test is performed, the measured results are merged into a results file where this file is then used by the clinician application following the completion of the recipient session. This file can be exported to the clinician in a number of ways, for example the file can be sent via the Internet or LAN connection to the recipient where it is received by the clinician application and acted upon. Or it can be stored in the recipient's speech processor and accessed from the speech processor at the recipient's next visit to the clinician.

An example configuration file is shown below:

```
<?xml version=“1.0” encoding=“utf-8” ?>
<Configuration>
  <PatientID>AEB182FC-F435-4f7e-85AE-D0D262464D86</PatientID>
  <TestID>B7316444-052D-464c-BC68-DC6474B822AA</TestID>
  <TestItems>
```

<Test1>

<Name>Impedance Test</Name>
<Channels>

<Channel>1</Channel>
<Channel>5</Channel>

</Channels>
<Modes>

<Mode>CG</Mode>
<Mode>MP1</Mode>

</Modes>

</Test1>
<Test2>

<Name>Threshold Level Measurement</Name>
<Map>1</Map>
<Channels>

<Channel>1</Channel>
<Channel>5</Channel>
<Channel>10</Channel>

</Channels>

</Test2>

</TestItems>
</Configuration>

This example configures an impedance test (test 1) and a threshold level measurement test (test 2) on the recipient application. As can be seen, the impedance test is carried out on channels 1 and 5 of the recipient's hearing implant in both common ground (CG) and monopolar 1 (MP1) mode. The threshold level measurement test is carried out on channels 1, 5 and 10. Each of these parameters are set by the clinician when configuring the test as discussed in relation to FIGS. 3, 4 and 5.

Based upon this configuration file shown above, an example results file generated is also shown below.

```
<?xml version=“1.0” encoding=“utf-8” ?>
<Result>
  <PatientID>AEB182FC-F435-4f7e-85AE-D0D262464D86</PatientID>
  <TestID>B7316444-052D-464c-BC68-DC6474B822AA</TestID>
  <TestItems>
```

<Test1>

<Name>Impedance Test</Name>
<Impedance>

<Mode>CG</Mode>
<Channel>1</Channel>
<Value>10500</Value>

</Impedance>
<Impedance>

<Mode>CG</Mode>
<Channel>5</Channel>
<Value>10560</Value>

</Impedance>
<Impedance>

<Mode>MP1</Mode>
<Channel>1</Channel>
<Value>10900</Value>

</Impedance>
<Impedance>

<Mode>MP1</Mode>
<Channel>5</Channel>
<Value>10800</Value>
</Impedance>

</Test1>
<Test2>

<Name>Threshold Level Measurement</Name>
<Level>

<Channel>1</Channel>
<Value>105</Value>

</Level>
<Level>

<Channel>5</Channel>
<Value>115</Value>

</Level>
<Level>

<Channel>10</Channel>
<Value>106</Value>

</Level>

</Test2>

</TestItems>
</Result>

As is shown above, the results of the configured tests, test 1 and test 2, are merged into this results file.

Following collection of the data for each test, the results data is then sent back to the clinician for evaluation and decision on what course of action is required.

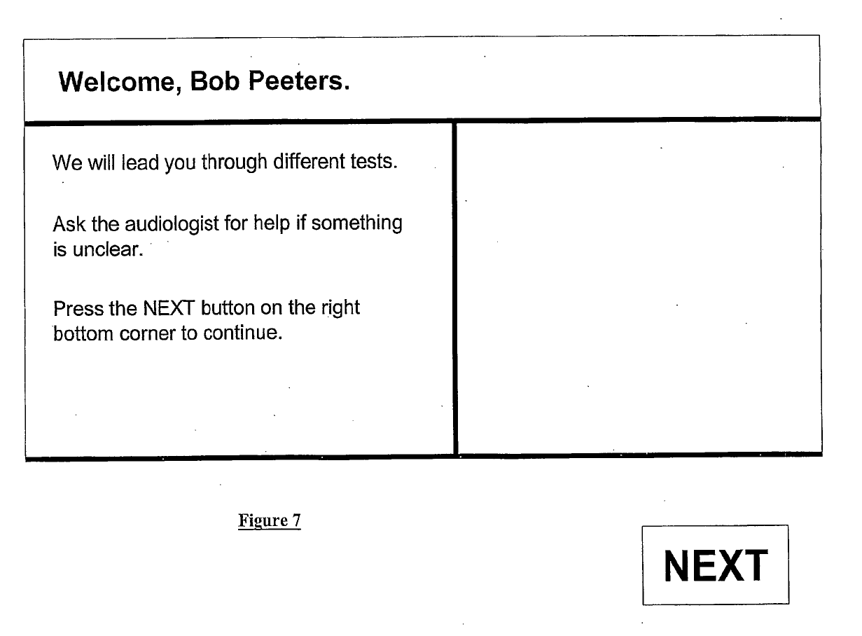

An example recipient interface that would appear on the recipient's computer 4 running the test configuration prepared in FIG. 4 will now be described in relation to FIGS. 7-14.

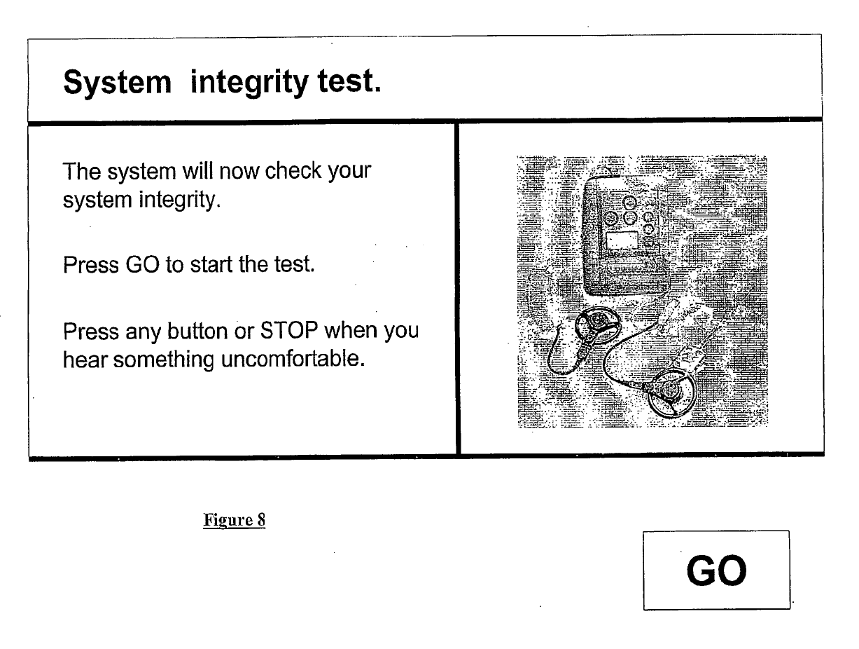

As shown in FIG. 7, once the recipient has received the configured test from the clinician, the interface runs a personal welcome page to the recipient wherein the recipient can be satisfied that the test is specifically designed for their own needs. Once the recipient proceeds by clicking “Next”, the recipient is informed, as depicted in FIG. 8, that the system will perform a system integrity check, to check that each aspect of the system is working correctly. The recipient is warned before any testing and in order for the test to be performed that recipient must elect to proceed with the test by clicking on the icon having the word “Go”.

It is worth noting on FIG. 8 that should the recipient experience any difficulties or discomfort during testing, they have the ability to stop the testing at any time by pressing any button or “Stop”. This feature is similar to what would occur in a regular clinical visit, where the recipient could indicate to the clinician any issues and the clinician would then stop the testing.

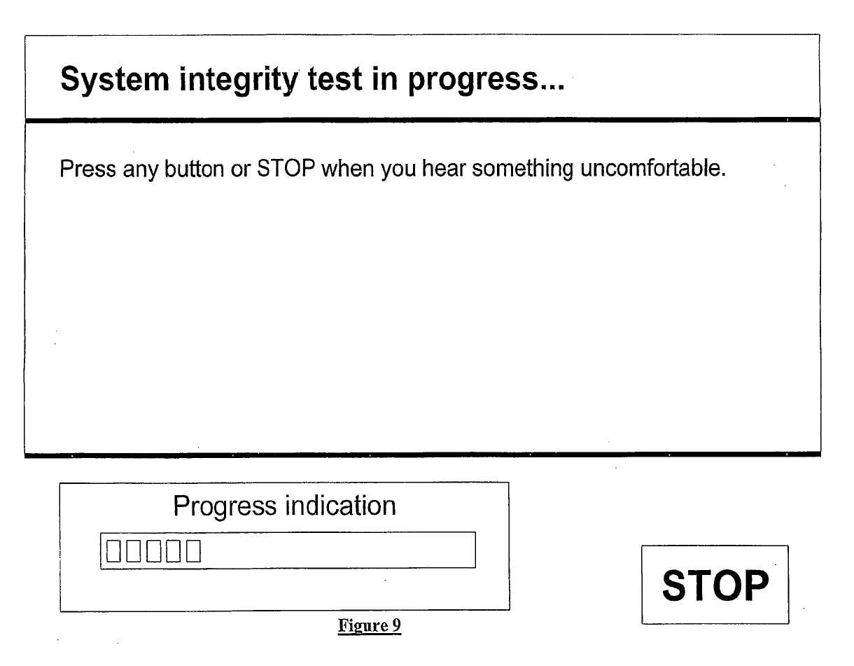

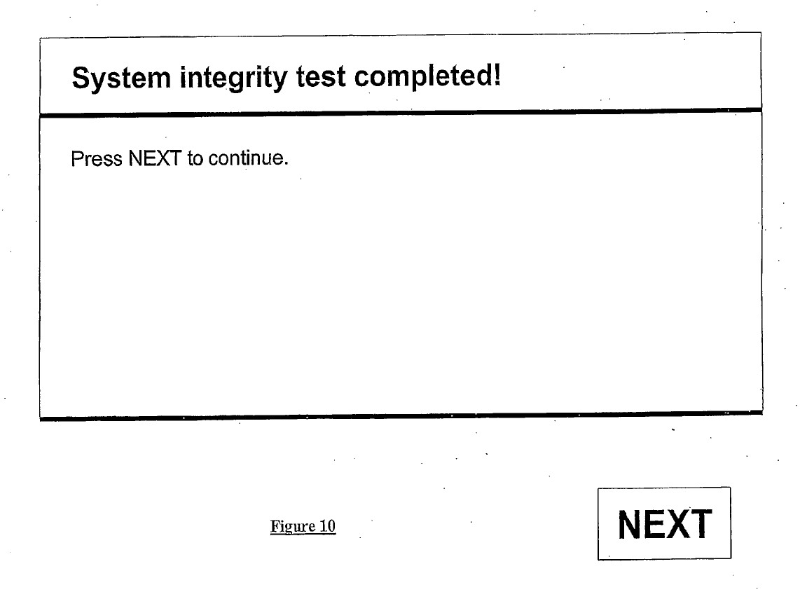

Once the recipient has started the system integrity test, the recipient is informed that the test is being performed and is provided with an indication of the progress of the test, see FIG. 9. Upon completion of the test, the recipient is informed that the test has been completed and is asked to proceed to the next stage in the process, see FIG. 10, by clicking the icon having the word “Next”.

At the completion of the system integrity test, the recipient application undergoes a basic test that checks whether the system integrity test has found any severe system integrity problems. Should such problems be found, the application would inform the recipient of this and suggest that the recipient contact the clinician for further attention. This would then bring a halt to the testing process, as the presence of system integrity problems would typically reduce the benefit of obtaining any further test results.

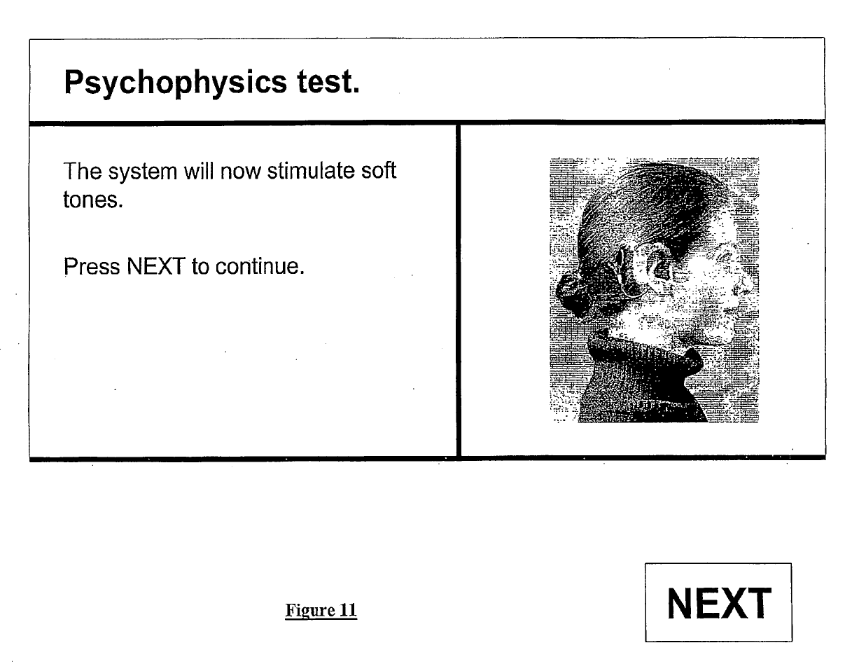

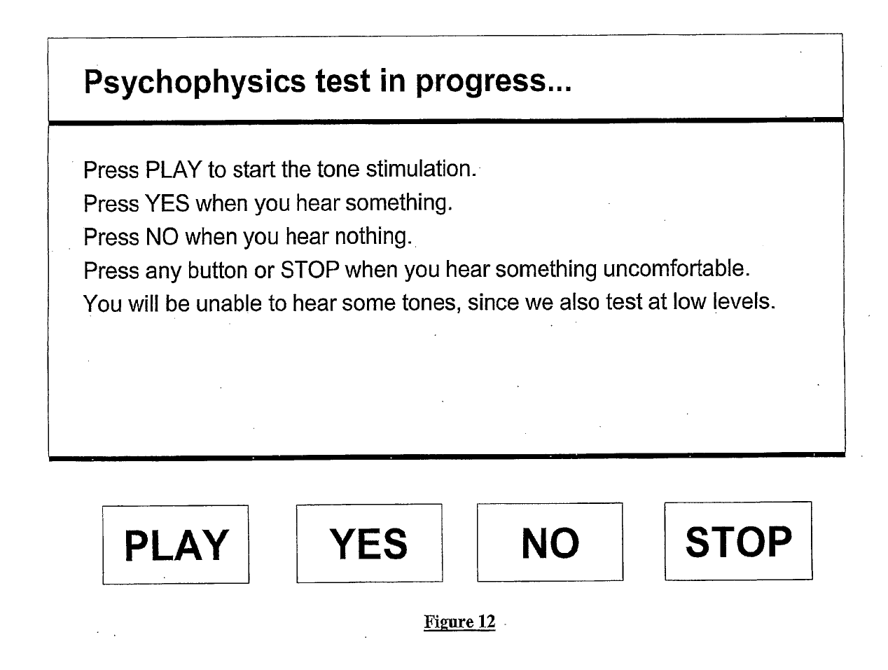

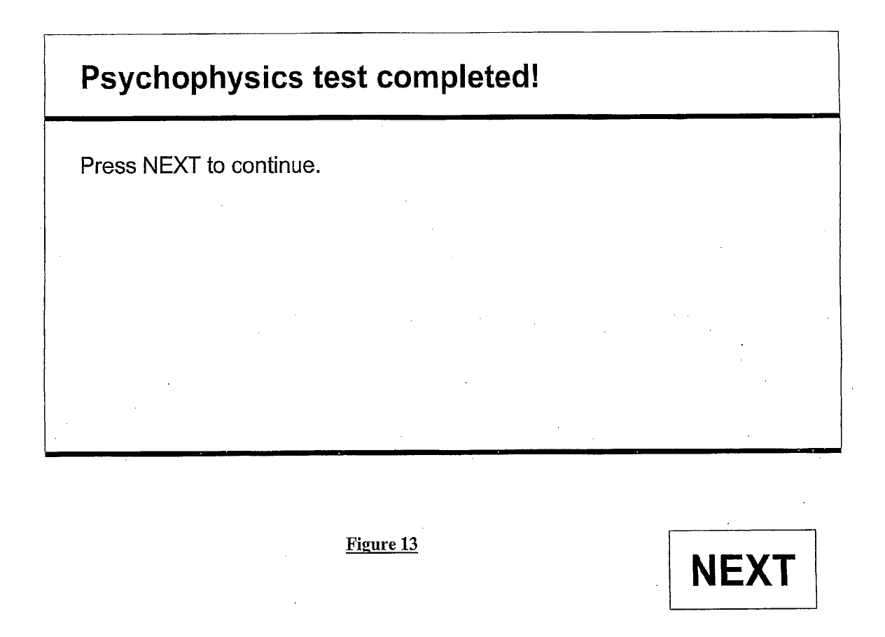

Following the successful completion of the system integrity test, the recipient is then informed of the next test in the process, namely a psychophysics test, see FIG. 11. Upon commencement of this test, the recipient is given instructions on how the test will be conducted and direction as to how input is required from the recipient. Typically, the input will be in the form of using a mouse to activate the appropriate icon on the computer screen. In the test as shown in FIG. 12, the recipient is asked to press YES when they hear a tone and NO when they do not hear anything. Once this test is complete, the recipient is informed of this and asked to proceed to the next step in the process by pressing “Next” (see FIG. 13).

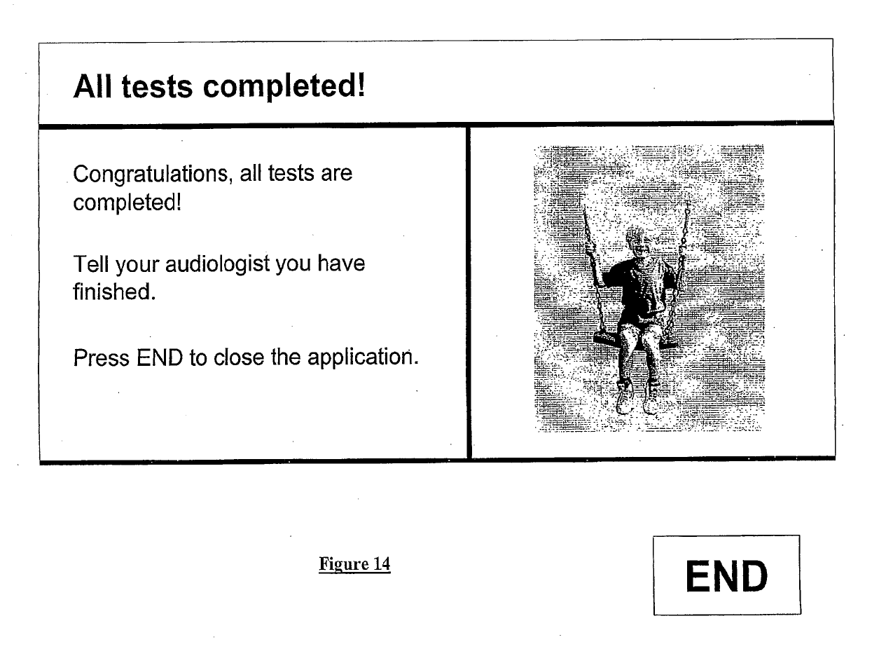

As shown in FIG. 14, when the recipient has completed all tests configured for them, they are informed of this and the application is closed. The recipient presses “End” to close the programme on their computer.

As discussed in relation to FIG. 2, upon completion of the configured tests, the results are sent to the clinician for evaluation. This is done by the clinician application which accesses the results file and tests the results in relation to previous or desired results.

An example of how this is done is discussed in the process below. This evaluation is related to the results of a T-NRT test performed on each of the recipient's electrodes as set by the clinician.

1. The T-NRT measurements, T-NRTn for all 22 electrodes of a recipient are stored in the results file following testing by the recipient. These results can be considered as T-NRTₙ(E1), T-NRTₙ(E2) . . . T-NRTₙ(E22).

2. The results of previously measured T-NRT measurements T-NRTₙ₋₁are also stored in the clinician application.

3. The differences between the current measurements, T-NRTₙ and the previously stored measurements, T-NRTₙ₋₁ are then obtained and compared for each electrode. The clinician would then set a limit as to the difference between the two which is considered acceptable, for example a clinician may consider that a difference of less than 10 current levels is acceptable for all electrodes.

Evaluation:
|T-NRTₙ(E1)−T-NRTₙ₋₁(E1)|<10 & |T-NRTₙ(E2)−T-NRTₙ₋₁(E2)|<10 . . . |T-NRTₙ(E22)−T-NRTₙ₋₁(E22)|<10

If the evaluation is positive, the application provides feedback to the clinician that the test is “OK” otherwise the feedback is “NOT OK”.

As shown in FIG. 15, upon an evaluation of the results as discussed above for all the tests undertaken, the clinician is provided with an interface providing an overview of the results. The overview as shown in FIG. 15 represents the results of the test carried out in FIGS. 7-14. As is shown, the test revealed that the recipient's system passed the system integrity test but failed the test of the recipient's threshold levels.

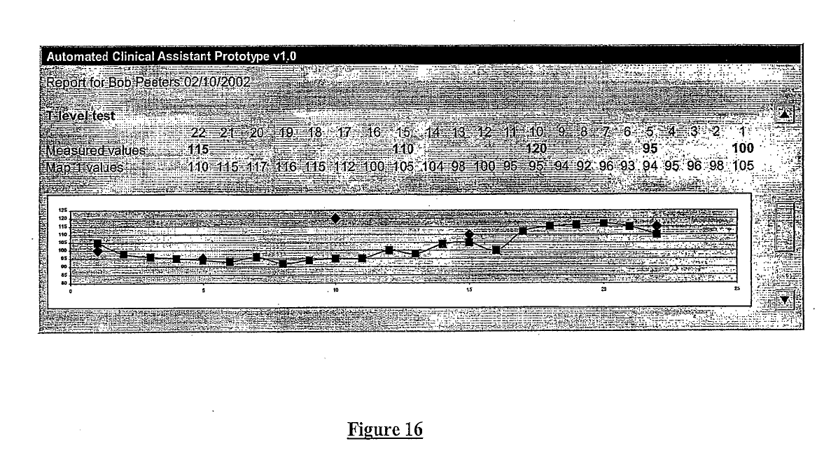

The clinician can then decide to view a detailed report of the test, which is shown in FIG. 16. This detailed report shows the measured values obtained in the test against the mapped values, or previously measured values in both a data form and a graphical form. As is evident from this detailed report, the majority of the measured results fall within an acceptable range of the previous results, except at electrode 10, where the difference is quite substantial.

Based upon this result the clinician can decide to proceed in a number of ways. Obviously, if the process indicated that all tests were passed, no specific action would be required and the clinician would inform the recipient of this and ask the recipient to repeat the test in, for example, 6 months time. Should there be some inconsistencies in the results, such as that shown in FIG. 16, the clinician may wish to see the recipient personally or send the recipient further tests to undertake to determine whether the inconsistency is merely an error in recordings or is a concern that requires further monitoring.

As can be appreciated, the present invention provides a system which enables faster, more user friendly and less skill-intensive follow-up care and management of a hearing implant. This system allows for personal expert care from clinicians and audiologists without the need to onerous clinician visits, and allows the recipient to interact with the clinician from their own home or school.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

1. A system for performing one or more tests on a hearing prosthesis, the system being usable at least in part by the recipient of the prosthesis, the system comprising:
a computer that processes software instructions and outputs signals in response to said instructions;
a prosthesis interface means that provides transfer of signals from said computer to the prosthesis and/or from the prosthesis to the computer; and
an interface that allows the recipient of the prosthesis to at least partially control at least some aspect of the tests performed on the prosthesis that is interfaced with the computer.

2. The system of claim 1 wherein the computer is a stand alone computer adapted to perform one or more tests on the hearing prosthesis.

3. The system of claim 1 wherein the computer is located in a clinic for use by the recipient when visiting the clinic.

4. The system of claim 3 wherein the computer is operable to provide results of the tests for immediate assessment by a clinician.

5. The system of claim 3 wherein the computer is operable to store results of the tests for later assessment.

6. The system of claim 1 wherein the computer is at a location remote from a clinician of the recipient.

7. The system of claim 6 wherein the computer is located at the recipient's home.

8. The system of claim 6 wherein the computer is operable to store results of the tests on a storage means for forwarding to the clinician of the recipient.

9. The system of claim 8 wherein the storage means is in the prosthesis.

10. The system of claim 9 wherein the storage means is a portable storage means, to be physically forwarded to the clinician of the recipient.

11. The system of claim 10 wherein the portable storage means is a magnetic disc.

12. The system of claim 1, wherein the computer is operable to obtain software instructions from the hearing prosthesis.

13. The system of claim 1 wherein the computer is operable to deliver results of the test electronically to a computer of a clinician of the recipient.

14. The system of claim 13 wherein the electronic delivery is provided by the Internet.

15-34. (canceled)

35. A method of testing a hearing prosthesis, the method comprising:
providing a first computer that processes software instructions and outputs signals in response to said instructions;
transferring the signals from the first computer to the prosthesis; and
providing an interface that allows the recipient of the prosthesis to at least partially control at least some aspect of the tests performed on the prosthesis that is interfaced with the first computer.

36-61. (canceled)

62. A computer program configured to make a computer perform one or more tests on a hearing prosthesis, the computer program being operable at least in part by the recipient of the hearing prosthesis, the computer program comprising:
code for generating instructions to the prosthesis in carrying out a test;
code for transferring the instructions to the prosthesis;
code for implementing an interface that allows the recipient of the prosthesis to at least partially control at least some aspect of the tests performed on the prosthesis.

63-83. (canceled)
